(19) United States
(12) Patent Application Publication
(10) Pub. No.: US 2010/0071095 A1
(43) Pub. Date: Mar. 18, 2010

(54) Method for Generating Plant Diversity By Incorporation of Microsatellite Sequences Into the Plant Genome

(76) Inventors: Iris Bahir, Haifa (IL); Shmuel Ben-Sasson, Jerusalem (IL)

Correspondence Address: LAW OFFICES OF ALBERT WAI-KIT CHAN, PLLC, 141-07 20TH AVENUE, WORLD PLAZA, SUITE 604, WHITESTONE, NY, 11357, US

(21) Appl. No.: 12/557,568

(22) Filed: Sep. 11, 2009

Related U.S. Application Data
(63) Continuation of application No. 10/550,870, filed on Jul. 24, 2006.

(30) Foreign Application Priority Data
Mar. 27, 2003 (IL) 155137

(51) Int. Cl. C12N 15/82; A01H 5/00

(52) U.S. Cl. 800/292; 800/278; 800/298

(57) ABSTRACT

The present invention provides a method for the generation of genetically diverse plants via the incorporation of micro-satellite (MS) sequences into the planar genome. The method involves obtaining MS-like DNA fragment, introducing said DNA into plant cells, and selecting said cells for the incorporation of the DNA. Finally, plants are grown from the selected cells. Said plants display phenotypic variability, and are more diverse than the parental plant. Using the method herein presented, novel plant varieties are generated.

### FIELD OF THE INVENTION

The present invention relates to the field of genetic engineering of plants. More specifically, the present invention provides a method for the generation of diverse new plant varieties, using micro-satellite sequences as a tool to achieve plant diversity.

### BACKGROUND OF THE INVENTION

All publications mentioned throughout this application are fully incorporated herein by reference, including all references cited therein.

Micro-satellites (MS) are repetitive DNA sequences typified by a monotonous repetition of short DNA sequences of between one to about ten nucleotides in length of the repeating unit [Moxon E. R. and Wills C. (1999) Scientific American 280(1):72-7]. The following DNA stretches are examples of MS sequences, (A)n, (CA)n, (CAG)n, (GATA)n, (TAGAAA)n, where n can vary between 3 to about 100 and the total length of the MS varies between about 10 to about 200 base-pairs (bp) in length. MS sequences may contain errors in the form of a base change or a missing nucleotide (i.e. a point mutation or a frame-shift) but still are considered hereby as genuine MS-like sequences. MS are generally considered as “junk DNA”, a remnant of Darwinian evolution that the genome could not get rid of.

Until now, conventional genetic engineering of plants target specific genes that are advantageous from a commercial point of view. For example, generation of transgenic plants that are resistant to herbicides or plants that produce fruits with a protein of higher nutritional value [McLaren J. S. (1998) Pest Outlook 12:36-41]. This is usually done via the introduction of foreign genetic elements into the plant genome [Kumar S. and Fladung M. (2001) Trends in Plant Science 6:155-9]. However, many of the important traits of plants are quantitative in nature and depend on the level of expression of multiple genes. Therefore manipulating the expression of only one gene may not be as effective in actually achieving the desired phenotype.

The present invention is based on the premise that MS sequences have a regulatory role in the plant genome. Thus, the introduction of MS sequences into the plant genome might result on a profound effect on the pattern of gene expression in the manipulated plant. It is an object of the present invention to provide a method whereby the incorporation of selected MS sequences into the genome of a large number of plant cells, and the growth of individual plants out of these individual cells, generates a plethora of new plant varieties.

These and other objects of the present invention will become apparent as the description proceeds.

### SUMMARY OF THE INVENTION

In a first aspect, the present invention relates to a method for the generation of genetically diverse plants via the incorporation of exogenous micro-satellite (MS) sequences into the plant genome, wherein said plants are of the same species, and said method comprises the following steps:
(a) obtaining MS-like DNA fragments;
(b) introducing said DNA fragments into plant cells;
(c) selecting the plant cells containing said DNA fragments;
(d) cultivating the plants grown from the selected cells under suitable conditions.

Optionally, the MS-like DNA fragments obtained in step (a) may be ligated into suitable vectors and then introduced into plant cells.

In a preferred embodiment, the MS-like DNA fragment (also referred to as exogenous MS, or exogenous DNA) is introduced into plant cells concomitantly with a selective marker. Said selective marker may be a gene that confers resistance to an antibiotic, a herbicide or a metabolic inhibitor.

In one embodiment of the method of the invention, the MS-like DNA fragment comprises a monotonous repeat of one to six nucleotides and is at least twelve nucleotides in length, wherein said repeat is any one of A/T, AT/TA, AG/CT, AAG/CTT, CGG/CCG, ATCG/CGAT, AAAT/ATTT, AAGTTC/GAACTT, CTG/CAG, TTTA/TAAA, CT/AG and TTC/GAA.

In another embodiment, the MS-like DNA fragment comprises a sequence that is at least 70% homologous to the above-mentioned monotonous repeat.

In a further embodiment of the method of the invention, the synthetic MS-like DNA fragment further includes in tandem a unique identifiable sequence that enables specific tagging of the incorporated DNA.

In a yet further embodiment of the invention, the synthetic MS-like DNA fragment is introduced into individual plant cells. Alternatively, the synthetic MS-like DNA fragment is introduced into any one of a plant embryo, a plant tissue or callus, or a leaf, which are then subsequently disintegrated into individual plant cells. Said individual cells are cultivated to give rise to individual plants.

In a further embodiment of the method of the invention, the exogenous DNA is obtained via synthesis or cloning. In addition, the exogenous DNA may be produced by the ligation of several DNA pieces.

In a last embodiment of the method of the invention, the DNA may be introduced via any one of electroporation, chemical or mechanical means, or liposomes. Said DNA may be either naked, or it may be comprised within a construct, and be introduced into the plant genome by a genetic vehicle such as a plasmid or a viral vector.

In another aspect, the invention refers to the use of MS-like DNA fragments as a tool for the generation of new plant varieties, or for the generation of any one of cells, seeds or progeny of said plants.

In a further aspect, the invention provides a plant variety produced by the method of obtaining and introducing MS-like DNA fragments into plant cells, selecting the plant cells containing the exogenous DNA, and cultivating the plants grown from said selected cells under suitable conditions.

A further aspect of the invention refers to a plant variety whose genome has been modified by the method described in the present invention.

Lastly, the present invention provides a new plant variety generated by the introduction of MS-like DNA fragments into its genome, and cells, seeds and progeny thereof.

### BRIEF DESCRIPTION OF THE FIGURES

FIG. 1: Flowchart summarizing the invention

FIG. 2A-D: Generation of novel plant varieties via microsatellite insertion.

FIG. 2k Dwarf plants.

FIG. 2B: Plant with malformed leaves.

FIG. 2C: Plant with narrow leaves.

FIG. 2D: Plant with short internodes.

FIG. 3: New variant phenotype (1).

Left—Wild-type plant.

Right—New plant variety carrying the micro-satellite (CTG)n: short plant with narrow twisted leaves.

FIG. 4: New variant phenotype (2).

Left—Wild-type plant.

Right—New plant varieties carrying the micro-satellite (TTTA)n: Dwarf plant (center) and plant with distorted leaves (far right).

FIG. 5: New variant phenotype (3).

Left—Wild-type plant.

Right—New plant variety carrying the micro-satellite (CT)n: Giant plant.

FIG. 6: New variant phenotype (4).

Left—Wild-type plant.

Right—New plant variety carrying the micro-satellite (CT)n: Dwarf plant.

FIG. 7: New variant phenotype (5).

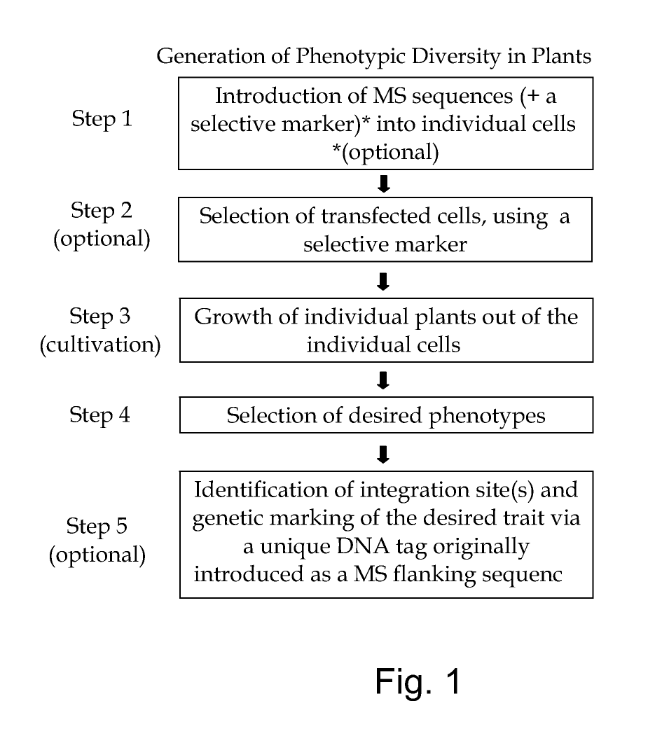

Left—Wild-type plant.

Right—New plant variety carrying the micro-satellite (TTC)n: Plant with distorted leaves.

### DETAILED DESCRIPTION OF THE INVENTION

In search for a method for generating plant diversity, the present inventors based on the premise that MS sequences have a regulatory role in the plant genome, and that consequently, insertion of such DNA sequences into plant cells will result in diversified phenotype.

Thus, in a first aspect, the present invention provides a method for the generation of genetically diverse plants via the incorporation of exogenous micro-satellite (MS) sequences into the plant genome, wherein said plants are of the same species, and said method comprises the following steps:
(a) obtaining MS-like DNA fragments;
(b) introducing said DNA fragments into plant cells;
(c) selecting the plant cells containing said DNA fragments;
(d) cultivating the plants grown from the selected cells under suitable conditions.

As shown in the following examples, MS DNA fragments were obtained through synthesis, using a DNA synthesizer.

As for the selection step, as shown in the following examples, MS sequences may be co-transfected with a gene that confers resistance to the antibiotic kanamycin. The cells are then grown on kanamycin-containing medium, and thus only those cells that were transfected survive.

The incorporation of MS sequences into new sites within the plant genome enables the generation of a number of plant strains with a broad diversity of characteristics that is genetically inherited. The desired strain shall then be selected according to preferred properties. For example, plants can be selected for flower or fruit size, plant height, resistance to herbicides, endurance to salinity or heat, or any other feature that might be advantageous for different purposes. Thus, the inventors present a novel approach for genetic engineering of plants and a method to its implementation.

As shown in Example 3, the introduction of the MS fragment made by the repeat (TTA)n, or by the repeat (CT)n generated more than one different plant variety. This shows that transfection with even one MS type, resulted in a collection of variants, probably due to the random nature of MS integration into the genome. In other words, since the MS sequences integrated at different sites in the genome of different plant cells, the phenotypic outcome is different and is manifested by the generation of a spectrum of variants. Nevertheless, each variant is genetically determined, and its phenotype should therefore be genetically inheritable.

When introduced into the plant cells, the DNA fragments may be carried by genetic vectors or not, and the site of the integration shall be random.

In one embodiment of the method of the invention, the DNA fragments may be ligated to suitable vectors and then introduced into the plant cells.

In another embodiment, the MS sequence utilized in the method of the invention comprises a monotonous repeat of one to six nucleotides, at least twelve nucleotides long, with maximum length of 10,000 nucleotides. In general, the repeat will be between about 70 and 120 nucleotides long, preferably between about 80 and 110 nucleotides, more preferably between 90 and 100 nucleotides long.

The repeat may be any one of A/T, AT/TA, AG/CT, AAG/CTT, CGG/CCG, ATCG/CGAT, AAAT/ATTT, AAGTTC/GAACTT, CTG/CAG, TTTA/TAAA, CT/AG and TTC/GAA. Preferably, said repeat is any one of AAGTTC/GAACTT, CTG/CAG, TTTA/TAAA, CT/AG and TTC/GAA. These repeats are denoted herein as in double stranded DNA, i.e. the 5′-3′/3′-5′ sequence.

In the herein presented examples, the following MS-like DNA fragments, of around 90 base-pairs full length, containing the repeats (AAGTTC/GAACTT)₁₅ (SEQ. ID. No.1), (CTG/CAG)30 (SEQ. ID. No.2), (TTTA/TAAA)₂₂ (SEQ. ID. No.3), (CT/AG)₄₅ (SEQ. ID. No.4), and (TTC/GAA)₃₀ (SEQ. ID. No.5) were used, and their respective full-length sequences are as follows:

SEQ. ID. No. 1:
AAGTTCAAGTTCAAGTTCAAGTTCAAGTTCAAGTTCAAGTTCAAGTTCAA
GTTCAAGTTCAAGTTCAAGTTCAAGTTCAAGTTCAAGTTC
SEQ. ID. No. 2:
CTGCTGCTGCTGCTGCTGCTGCTGCTGCTGCTGCTGCTGCTGCTGCTGCT
GCTGCTGCTGCTGCTGCTGCTGCTGCTGCTGCTGCTGCTG
SEQ. ID. No. 3:
TTTATTTATTTATTTATTTATTTATTTATTTATTTATTTATTTATTTATT
TATTTATTTATTTATTTATTTATTTATTTATTTATTTA
SEQ. ID. No. 4:
CTCTCTCTCTCTCTCTCTCTCTCTCTCTCTCTCTCTCTCTCTCTCTCTCT
CTCTCTCTCTCTCTCTCTCTCTCTCTCTCTCTCTCTCTCT
SEQ. ID. No. 5:
TTCTTCTTCTTCTTCTTCTTCTTCTTCTTCTTCTTCTTCTTCTTCTTCTT
CTTCTTCTTCTTCTTCTTCTTCTTCTTCTTCTTCTTCTTC

Alternatively, the MS-like DNA fragment comprises a sequence that is at least 70% homologous to said monotonous repeat, or fragments or derivatives thereof. Therefore, said DNA fragment may also comprise a sequence that is between 75 and 95% homologous to the repeat, or 80% to 90% homologous to the repeat. Homology between the DNA fragments may be evaluated through sequence comparison, using bioinformatics methods known to the man skilled in the art.

In another embodiment of the invention, the exogenous MS is introduced concomitantly with a selective marker, wherein preferably said selective marker is a gene that confers resistance to an antibiotic, a herbicide, or a metabolic inhibitor. Alternatively, the selective marker can be a gene that highlights the transfected cells such as the gene for green fluorescent protein (GFP). Thus, the MS-like DNA fragments and the selection marker will be introduced into the plant or plant cells by co-transfection. Preferably, said selective marker is the gene for kanamycin resistance.

In order to enhance the selection of cells that actually incorporated the transfected DNA, it is advantageous to introduce a selective marker concomitantly with the desired DNA fragment. This can be achieved by co-transfecting the cells with a genetic element that confers resistance to a toxic agent, like a herbicide or an antibiotic, that can kill non-protected cells which did not incorporate the exogenous DNA. For example, such a selection marker can be a gene for an enzyme that degrades the said herbicide or antibiotic or clones that highlight the transfected cells. Clones of such genes are well known to those versed in the art [for example see Zhang CL. et al. (2001) Mol. Biotechnol. 17:109-17 and references therein] and are commercially available. In addition, the introduced MS sequence can be flanked by a unique DNA sequence in order to tag the exogenously introduced MS. This shall enable an unequivocal identification of the site(s) of integration of the MS in the plant genome and the identification of a novel trait.

Once introduced into the cell nucleus, DNA fragments become incorporated into the plant genome. For reasons of simplicity, the term transfection is herein used to include all different methods of introduction of exogenous DNA into the plant genome.

Preferably the synthetic MS sequence may further include a unique identifiable sequence in tandem, which will enable specific tagging of the incorporated DNA and identification of the site of integration. This embodiment is particularly important for the unequivocal identification of the transgenic plants.

In a further embodiment of the invention, the synthetic MS sequence is introduced into individual plant cells. Alternatively, the synthetic MS sequence is introduced into any one of a plant embryo, a plant tissue, a callus, leaves, or any plant part where it is possible to introduce DNA. When the MS sequence is introduced into a plant cell which is part of a multi-cellular tissue, said tissue will be subsequently disintegrated into individual plant cells, to obtain a single cell suspension. The cells containing the introduced DNA will be selected, and then cultivated under suitable conditions to give rise to individual plants.

Plant cells can be transfected with exogenous DNA while part of a plant embryo, plant tissue like a leaf, a wound tissue or non-differentiated callus, or can be transfected while in single cell suspension or as protoplasts (plant cells whose cell-wall was dissolved). Following DNA transfection, selection of the modified cells is made at the single cell level by using an incorporated selectable marker screening procedure, which is then followed by development into individual plants.

The MS sequences to be incorporated into the plant genome may be introduced via any technique known to the artisan for the introduction of DNA sequences into a genome, such as electroporation, mechanical means like the gene gun (also known as particle bombardment), chemical means such as polyethylene glycol, or by the use of liposomes, with or without being carried by vectors. Alternatively, the DNA may be introduced into protoplasts. Preferably, the MS-like DNA fragments are introduced via mechanical means, like the gene gun.

The MS sequences may be introduced into the plant genome as fragments. Alternatively, these sequences may first be ligated. Such a ligated construct may be further incorporated into a genetic vehicle such as a plasmid or a viral vector, and then introduced into the plant genome.

The exogenous MS sequences to be used in the method of the invention are to be obtained via synthesis or cloning, i.e., said MS-like DNA fragments may be synthetically made or they may be isolated from the genome of a plant cell.

In an additional embodiment of the invention, the exogenous DNA construct to be introduced for the generation of plant diversity may be produced by the ligation of the MS-like fragments with a marker DNA, a tagging sequence and a vehicle, or any combination thereof. Thus, the MS-like fragment may be introduced as naked DNA or as part of a larger structure, as for example in a DNA construct.

As shown in Examples 1 and 2, an effective way of introducing the MS-like fragments into the plant cells is through the gene gun technique (particle bombardment).

The cultivation step of the method of the present invention may itself be selective. For example, plants may be cultivated under stressing conditions such as high salinity, for selection of salt-resistant plants, or be treated with herbicides, for selection of herbicide-resistant plants. Thus, the invention also encompasses the selection of plants with desired traits.

In a third aspect, the invention provides the use of MS sequences as a tool for the generation of new plant varieties and cells, seeds or progeny thereof. It is to be understood that the method of the invention will modify the genome of a very large number of plant cells at a single time. Since insertion of said MS sequences will occur at random, a very large number of new phenotypes will be generated by the method of the invention. Within such large choice of new phenotypes, beneficiary traits will be abundant.

Example 2 demonstrates how, from a relatively small number (total 36) of plants transformed with the MS sequence (CT)n, 50% presented a mutant phenotype, either dwarf or giant. Both giant and dwarf varieties of a plant may be desired. A giant plant may be beneficial for, for example, providing bigger fruits, or even for decorative purposes. A dwarf variety of a plant may also be desired, for example, for decorative purposes (being able to fit in an in-doors plant bed).

Another aspect of the invention is a plant variety produced by the method of the invention. The method of the invention shall enable the establishment of a diversity of stable phenotypes, from which new plant varieties may be developed.

A further aspect of the invention provides a plant variety whose genome has been modified by the method of the invention.

Lastly, the invention provides a new plant variety generated by the introduction of MS-like DNA fragments into its genome and cells, seeds and progeny thereof.

As shown in the following examples, the present invention provides some new varieties of the tobacco plant. A dwarf plant, a giant plant, a plant with deformed leaves, a plant with narrow twisted leaves, a short plant and a plant with short internodes were generated, and are presented as examples of generation of plant diversity.

Thus, the inventors hereby specify the incorporation of selected MS sequences into the genome of a large number of plant cells and the growth of individual plants out of these individual cells, by methods known to those versed in the art. Once integrated into the plant genome, this new pattern of MS distribution should be genetically stable and heritable.

The exact nature of the ensuing phenotypic change cannot be predicted in advance due to sporadic sites of MS integration and variability in copy number, unique to each transfected plant cell. Nevertheless, it should lead to the creation of a broad spectrum of phenotypes through the integration of MS sequences in different sites in the genome of different cells, occurred at random, as exemplified herein.

The method presented herein opens the way for the generation of genetically defined strains of desirable phenotypes, through the selection of a desired trait, like for example plant size, fruit size, flower shape and color, resistance to salinity or heat, amongst others.

In the following examples, the inventors used the tobacco plant as a model for establishing the method of the invention. It must be emphasized that the method of the invention may be applied to any plant species, amongst which crop plants like avocado, cacao, date palm, mango, mate, melon, papaya, tomato, cucumber, pepper, pineapple, strawberry, tea, coffee, sugar cane, cotton, rice, wheat, soybean, bean, canola, barley, onion, potato, carrot, garlic, ginger are a few examples. Similarly, the method of the invention may also be used for blooming plants like roses, carnations, lilies, or house plants like gardenia, amaryllis, Christmas cactus, cyclamen, to name but few.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

### Synthesis of DNA Fragments

DNA fragments can be synthetically made using commercially available DNA synthesizers [for details see: Current Protocols in Molecular Biology, Editors: Ausubel FM et al. (2002) Published by John Wiley & Sons], or via the conventional polymerase chain reaction (PCR) method [Ausubel et al. (2002) id ibid.]. The different DNA fragments (e.g the MS and the tagging sequence) are then ligated by methods known to those versed in the art.

The DNA fragments (micro-satellite sequences) used in the following examples were obtained synthetically, utilizing a DNA synthesizer.

Introduction of DNA Fragments into Plant Cells

A DNA can be transferred into plant cells by applying a high voltage, a method known as electroporation [D'Halluin K et al. (1992) Plant Cell 4:1495-1505], or by a temporary mechanical disruption of the cell membrane [Taylor N J and Fauquet C M. (2002) DNA Cell Biol. 21:963-77]. This last method is conventionally known as the gene gun, and involves coating miniature metal beads (gold or tungsten, around 1 μm in diameter and less) with the exogenous DNA and forcing them through the cell membrane using a high pressure device, known as particle bombardment gun. Yet another way to introduce exogenous DNA into the plant genome is via genetic vectors such as plasmids or viral genomes. Examples of such genetic vectors are Agrobacterium-mediated transformation, or the infection of cells with recombinant viruses such as the tobacco mosaic virus (TMV) or the cowpea mosaic virus (CPMV).

### Preparation of Tobacco Cell Culture for Transformation by Particle Bombardment

### Materials:

55 mm Petri dishes

Mesh 18 sieves, cut into 20 mm squares and sterilized

### Growing Media:

### 1) Cell Culture Medium (514): pH 5.87

MS macro minerals

MS micro minerals

10.3 mg/l Thiamine

9.5 mg/l Pyridoxine

4.5 mg/l Nicotinic acid

0.25 mg/l Kinetin

3.0 mg/l 2,4-D

0.2% Casein Hydrolysate

3.0% Sucrose

### 2) Regeneration Medium (1041): pH 5.8

MS macro minerals

MS micro minerals

MS vitamins

0.1 mg/l IAA

2.0 mg/l Zeatin

2% sucrose

1% Manitol

agar

### 3) Selection Medium:

Regeneration Medium 1041+50 mg/l Kanamycin

### 4) Rooting Medium: pH 5.8

MS macro minerals

MS micro minerals

MS vitamins

3% sucrose

agar

100 mg/l Kanamycin

### Method:

1) Prepare 60 mg microparticles according to the protocol below.
2) Choose as fine a cell suspension as possible, avoiding large cell agglomerates, for example, 10,000 cells/100 microlitre of medium.
3) Transfer the cell suspension to a sterile 50 ml test-tube and allow the cells to settle. Adjust the volume of the medium (514) in order to obtain a ratio of 1:1 cells:medium.
4) Resuspend all and transfer the cell suspension in 400-500 microlitre aliquots onto the sieve squares placed on Whatman paper. It is important to obtain as fine and uniform cell layer as possible.
5) Transfer the sieves and cells to Petri dishes containing regeneration medium (1041).
6) Clean the bombardment chamber thoroughly with alcohol and place prepared Petri dish in the center of the bombardment platform/dish. Distance of platform from syringe filter=9 cm.
7) Proceed with bombardment as described [Taylor and Fauquet (2002) id ibid.].
8) 7-10 days after bombardment transfer sieves and cells to selection medium (1041)+50 mg/l Kanamycin and renew medium weekly.
9) Plantlets with at least two leaves are transferred to rooting medium (88)+80 mg/l Kanamycin.

### Microcarrier Preparation:

(for 120 bombardments, using 500 μl microparticles for bombardment)
1. In 1.5 ml microfuge tube, weight 60 mg of microparticles.
2. Add 1 ml of 70% ethanol, vortex for 5 min
3. Incubate for 15 min (at room temperature).
4. Pellet the microparticles by spinning for 2-5 min.
5. Discard liquid. The following steps should be repeated 3 times:
Add 1 ml of sterile water.
Allow the particles to settle for 1 min
Pellet the particles by spinning for 90 sec. for the first time, 120 sec., for the second time and 150 sec. for third time.
   6. Remove the liquid and discard.
   7. Add 1 ml sterile 40% PEG, to bring the particles to 60 mg/ml concentration (assuming to lose during preparation).
   Coating DNA onto Microcarriers:
   (The following procedure is sufficient for six bombardments. When removing aliquots of microcarriers, it is important to vortex the tube containing the microcarrier continuously in order to maximize uniform sampling.)
   1. Vortex the microcarriers prepared in 40% PEG (60 mg/ml) for 5 min on a platform vortex, to resuspend and disrupt agglomerated particles.
   2. Remove 50 μl (3 mg) of microcarriers to 1.5 microfuge tube.
   3. While vortexing vigorously, add in order:
5 μl DNA (at approx. 1 μg/μl concentration)
50 μl Ca(NO3)
[20 μspermidine base (0.1M)].
   4. Continue vortexing for 2-3 min.
   5. Allow the microcarriers to settle for 1 min.
   6. Pellet the microcarriers by spinning for 2 seconds in a microfuge.
   7. Remove the liquid and discard.

### Example 1

### Generation of Phenotypic Diversity Via the Introduction of a Microsatellite Sequence into the Plant Genome

DNA of the micro-satellite sequence (AAGTTC)n of a total length of about 90 base-pairs (n˜15), was introduced into tobacco cells utilizing the particle bombardment method as specified above. The full-length sequence of this DNA fragment is denoted by SEQ. ID. No.1.

The tobacco cells to be transfected were obtained from a primary culture of tobacco cells obtained from leaves.

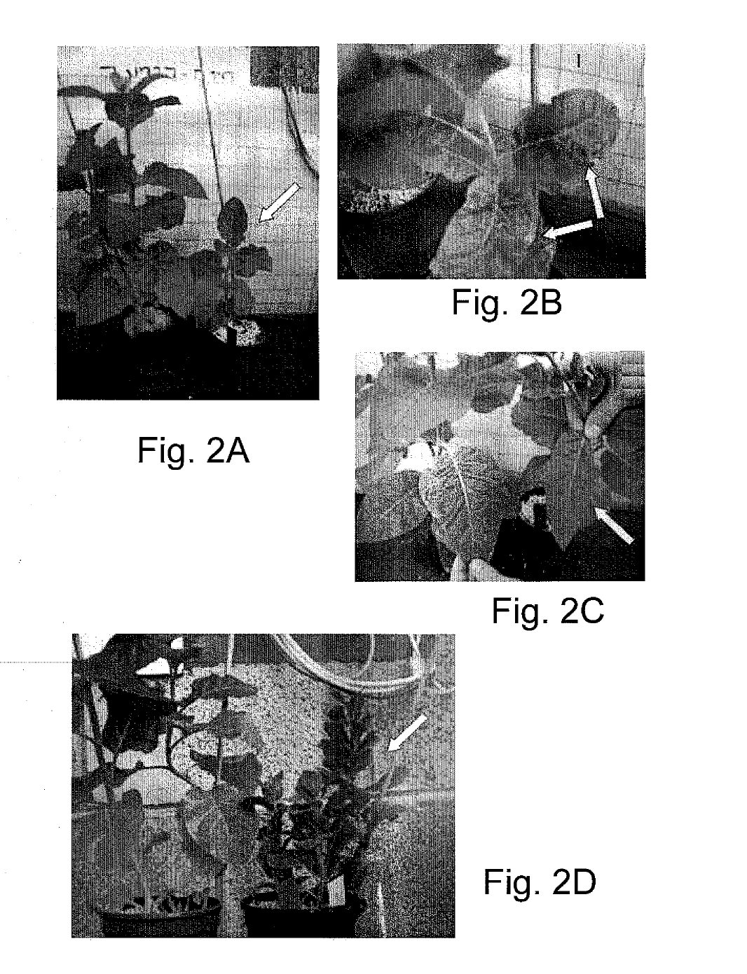

The transfected cells were then allowed to grow in a rooting medium and the developing plants were examined for phenotypic changes. As seen in FIG. 2, a considerable number of plants (26 of 116 total number of plants, 22.4%) displayed a consistent change in shape. 7 out of 116 plants (6%) presented a dwarf phenotype. 4 out of 116 plants (3.4%) presented malformed leaves. 5 out of 116 plants (4.3%) presented narrow leaves. 10 out of 116 plants (8.6%) presented short internodes, which were between 1 and 2 cm, as opposed to the length of the internodes in control plants, which was around 5 cm long.

### Example 2

### Generation of Phenotypic Diversity Via the Introduction of a Microsatellite Sequence into the Plant Genome(2)

The DNA of the below listed micro-satellite sequences of a total length of about 90 base-pairs, was introduced into tobacco cells utilizing the particle bombardment method as described above. Alternatively, the MS-like DNA fragments were ligated to a Ti plasmid and introduced into the cells via a Ti plasmid-containing Agrobacterium. The transfected cells were then allowed to grow in a rooting medium and the developing plants were examined for phenotypic changes.

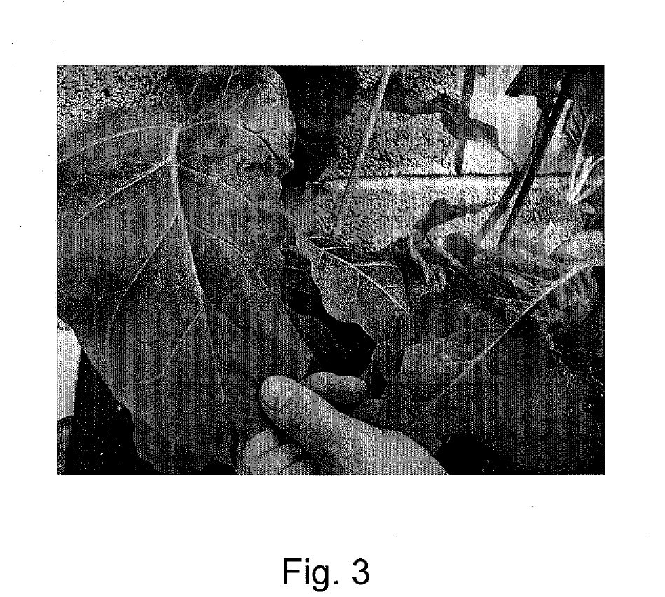

As seen in FIGS. 3 to 7, an array of phenotypes arose with the introduction of micro-satellites to the genome of the plants. A control, non-transfected plant, is displayed to the left of the figures, while the transfected variant is depicted to the right.

The following micro-satellite sequences were tested for their effect on the phenotype of Tobacco plants:

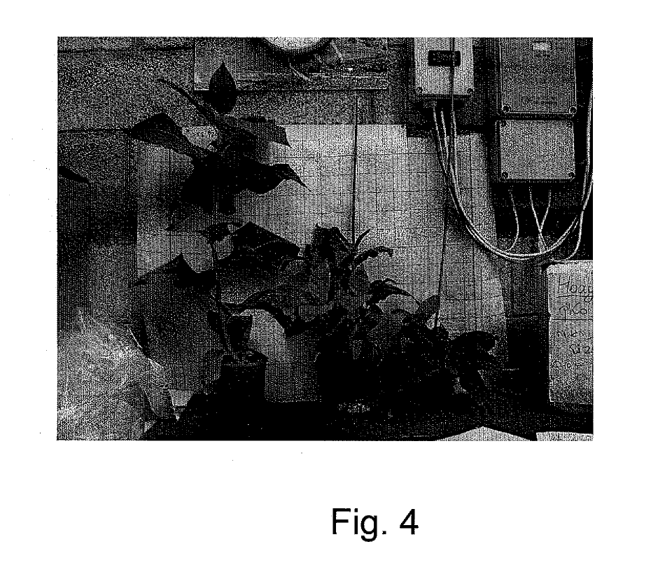

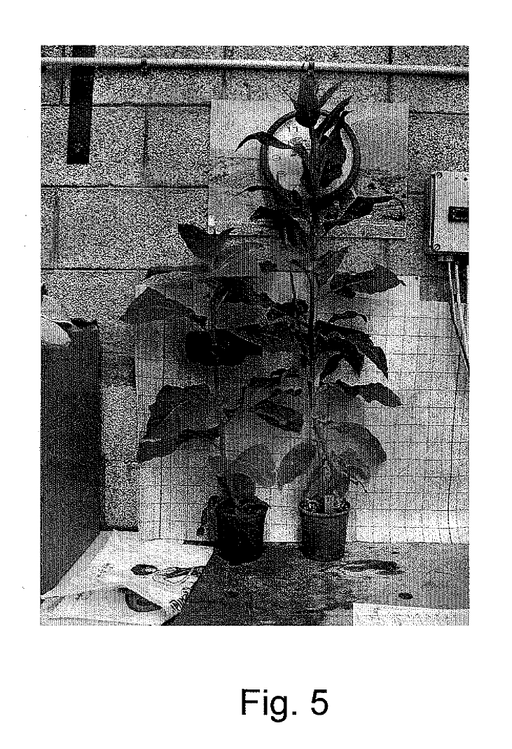

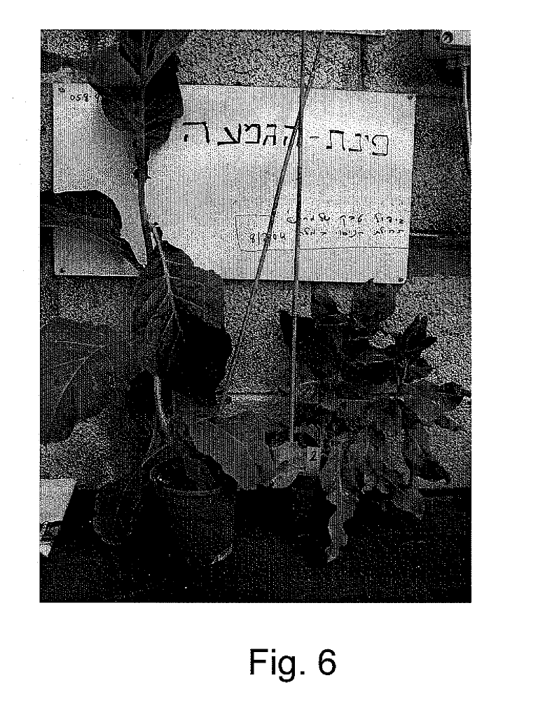

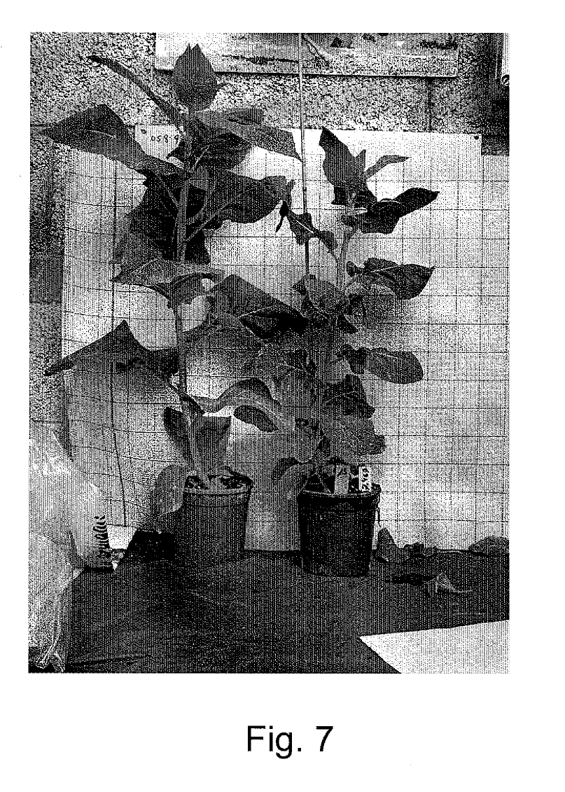

I. (CTG)n: This micro-satellite resulted in 9/10 mutants (90%), characterized by short plants with narrow twisted leaves (FIG. 3). The full-length sequence of the DNA fragment containing this repeat is denoted by SEQ. ID. No.2.
II. (TTTA)n: This micro-satellite resulted in 3/7 mutants (43%), characterized by dwarf plants (FIG. 4, center) and plants with distorted of leaves (FIG. 4, far right). The full-length sequence of the DNA fragment containing this repeat is denoted by SEQ. ID. No.3.
III. (CT)n: This micro-satellite resulted in 18/36 mutants (50%), characterized by giant (FIG. 5) and dwarf (FIG. 6) plants. The full-length sequence of the DNA fragment containing this repeat is denoted by SEQ. ID. No.4.
IV. (TTC)n: This micro-satellite resulted in 5/14 mutants (36%), characterized by plants with distorted leaves (FIG. 7). The full-length sequence of the DNA fragment containing this repeat is denoted by SEQ. ID. No.5.

In general, the normal height of a tobacco plant is between 60 and 67 cm. Transformation with the MS-like DNA fragment generated dwarf plants, up to around 28-30 cm tall, short plants, from 39 cm up to around 50 cm tall, and giant plants, from 89 cm tall, with specimens reaching 95 cm-100 cm.

1-24. (canceled)

25. A method for generating genetically diverse plants via incorporation of exogenous micro-satellite (MS) sequences into the plant genome, said method comprises the steps of:
(a) obtaining MS-like DNA fragments comprising a monotonous repeat of two to six nucleotides, wherein the DNA fragment has a length of about 70 to about 120 nucleotides long;
(b) introducing said DNA fragments into plant cells;
(c) selecting the plant cells containing said DNA fragments; and
(d) cultivating the selected plant cells, thereby generating genetically diverse plants.

26. The method of claim 25, wherein the repeat is selected from the group consisting of AT/TA, AG/CT, AAG/CTT, CGG/CCG, ATCG/CGAT, AAAT/ATTT, AAGTTC/GAACTT, CTG/CAG, TTTA/TAAA, CT/AG, and TTC/GAA

27. The method of claim 25, wherein the MS-like DNA fragments comprise sequences selected from the group consisting of SEQ ID NOs. 1-5.

28. The method of claim 25, wherein optionally the MS-like DNA fragments obtained in step (a) are ligated into vectors and then proceed to step (b).

29. The method of claim 25, wherein the MS-like DNA fragments are introduced concomitantly with a selective marker.

30. The method of claim 29, wherein the selective marker is a gene that confers resistance to an antibiotic, a herbicide or a metabolic inhibitor.

31. The method of claim 25, wherein the MS-like DNA fragments are introduced by a genetic vehicle selected from the group consisting of a plasmid and a viral vector.

32. The method of claim 25, wherein the generation of genetically diverse plants further comprises generation of one of cells, seeds or progeny of said plants.

33. A plant produced by the method of claim 25, and cells, seeds and progeny thereof, wherein the cells of the plant comprise MS-like DNA fragments comprising a monotonous repeat of two to six nucleotides, wherein the DNA fragment has a length of about 70 to about 120 nucleotides long.

34. The method of claim 25, wherein the MS-like DNA fragments are introduced via any one of electroporation, chemical means or liposomes.


SEQUENCE LISTING (5 sequences)

SEQ ID NO: 1 - DNA, 90 bp, Artificial sequence
  Microsattelite sequence
aagttcaagttcaagttcaagttcaagttcaagttcaagttcaagttcaagttcaagttcaagttcaagttcaagttcaagttcaagttc

SEQ ID NO: 2 - DNA, 90 bp, Artificial sequence
  Microsattelite sequence
ctgctgctgctgctgctgctgctgctgctgctgctgctgctgctgctgctgctgctgctgctgctgctgctgctgctgctgctgctgctg

SEQ ID NO: 3 - DNA, 88 bp, Artificial sequence
  Microsattelite sequence
tttatttatttatttatttatttatttatttatttatttatttatttatttatttatttatttatttatttatttatttatttattta

SEQ ID NO: 4 - DNA, 90 bp, Artificial sequence
  Microsattelite sequence
ctctctctctctctctctctctctctctctctctctctctctctctctctctctctctctctctctctctctctctctctctctctctct

SEQ ID NO: 5 - DNA, 90 bp, Artificial Sequence
  Microsattelite sequence
ttcttcttcttcttcttcttcttcttcttcttcttcttcttcttcttcttcttcttcttcttcttcttcttcttcttcttcttcttcttc